(19) United States
(12) Patent Application Publication
(10) Pub. No.: US 2019/0177737 A1
(43) Pub. Date: Jun. 13, 2019

(54) Genetically Modified Tobacco

(71) Applicant: MyMD Pharmaceuticals, Inc., Tampa, FL (US)

(72) Inventor: Jonnie R. Williams, Sarasota, FL (US)

(21) Appl. No.: 16/218,574

(22) Filed: Dec. 13, 2018

Related U.S. Application Data
(60) Provisional application No. 62/598,052, filed on Dec. 13, 2017.

(51) Int. Cl. C12N 15/82; A01H 6/82

(52) U.S. Cl. CPC C12N 15/8243; A01H 6/823

(57) ABSTRACT

A method of increasing the level of isomyosmine in a tobacco plant that includes genetically modifying the tobacco plant to induce the tobacco plant to produce a tobacco with increased levels of isomyosmine in the tobacco. Further, the tobacco may have decreased levels of nicotine. In some examples, the decreased levels of nicotine are the result of a second genetic modification to the tobacco plant.

### CROSS REFERENCE TO RELATED APPLICATION

This application claims priority under 35 U.S.C. § 119(e) to U.S. Application No. 62/598,052, filed Dec. 13, 2017, the entire contents of which are incorporated herein by reference.

### BACKGROUND

Nicotine is the primary basis for tobacco addiction and the tobacco industry has experienced a renewed interest in producing tobacco plants and tobacco products with reduced levels of nicotine. Efforts to reduce the level of nicotine content in tobacco have existed for much of the history of tobacco cultivation. Techniques such as plant breeding, as well as chemical and processing methods have been used to reduce nicotine levels in tobacco. Modern efforts include genetically modified tobacco plants that produce reduced levels of nicotine. An individual's desire, however, to achieve the same physiological effects experienced from smoking or using a tobacco product with conventional levels of nicotine have inhibited the success of reduced nicotine products.

It would be desirable to reduce the levels of nicotine in tobacco products while still providing an individual with the pleasurable effects associated with tobacco use. Described herein are tobacco products featuring reduced levels of nicotine and increased levels of isomyosmine. Increased levels of isomyosmine in tobacco products may be achieved by the genetic engineering of tobacco plants or by introducing isomyosmine into tobacco material during processing.

A reduced nicotine tobacco product that has an increased content of isomyosmine may provide an attractive alternative to conventional tobacco products and may provide a viable option for reducing or eliminating cravings for nicotine or dependence on nicotine.

### SUMMARY

In accordance with aspects disclosed herein, methods of reducing the levels of nicotine and increasing the levels of isomyosmine in a genetically modified tobacco plant are disclosed. In other aspects, methods of increasing the levels of isomyosmine in a tobacco plant by genetically modifying the tobacco plant in such a manner that induces the tobacco plant to produce tobacco with increased levels of the alkaloid isomyosmine, and the tobacco may have decreased levels of nicotine. In other aspects, the tobacco plant may also produce tobacco with decreased levels of nicotine. In other examples, the tobacco plant may include a first genetic modification that induces the plant to produce increased levels of isomyosmine in the tobacco, and a second genetic modification that induces the tobacco plant to produce tobacco with decreased levels of nicotine content.

In accordance with another aspect, a method of increasing the isomyosmine in a tobacco plant that is genetically modified is disclosed in which the genetically modified plant has a nicotine converter rate of at least 3% or greater. In other aspects, the tobacco produced by the disclosed method has a nicotine content of less than about 2 mg/g. In still other aspects, the disclosed method includes a tobacco with an isomyosmine content from about 0.001 mg/g to about 10 mg/g, from about 0.01 mg/g to about 10 mg/g, or from about 0.1 mg/g to about 100 mg/g.

In accordance with another aspect, a method of increasing the isomyosmine in a tobacco plant that is genetically modified is disclosed in which the genetically modified plant is a solanecea plant. In yet another aspect, the genetic modification to the tobacco plant induces an increased production of isomyosmine, and the increased production of the isomyosmine results in the increased levels of isomyosmine in the tobacco. In still other aspects, the genetically modified tobacco plant includes a second genetic modification that decreases the levels of nicotine in the tobacco. In another aspect, the second genetic modification inhibits the expression of the methylenetetrahydrofolate reductase (MTHFR) gene of the tobacco plant and the inhibition of the expression of the MTHFR gene induces an increased expression of the nicotine N-demethylase (CYP82E4) gene. The increased expression or overexpression of the CYP82E4 gene results in an increased nicotine-to-nornicotine conversion rate, and the increased nicotine-to-nornicotine conversion rate decreases the levels of nicotine in the tobacco.

In accordance with other aspects herein, compositions of tobacco with increased levels of isomyosmine and a reduced level of nicotine are disclosed. In other examples, the tobacco is produced from a genetically modified tobacco plant that induces the production of increased levels of isomyosmine. In still other aspects, the isomyosmine is synthetically added to the tobacco. In still other aspects, the composition of tobacco has decreased levels of nicotine as a result of a genetic modification that induces a tobacco plant to produce tobacco with lower levels of nicotine. In still other examples, the composition of tobacco is chemically treated or processed to reduce the level of nicotine.

In accordance with certain aspects disclosed herein, the composition of tobacco may be formed into a tobacco product such as a cigarette, cigar, pipe tobacco, smokeless tobacco, chewing tobacco, capsule, tablet, or lozenge.

In accordance with other aspects, compositions of tobacco with increased levels of isomyosmine, and reduced levels of nicotine are disclosed in which the tobacco is produced from a genetically modified plant, and the tobacco plant has a genetic modification that causes a nicotine converter rate of about 3.0% or more, or at least 3.0%. In another aspect, the tobacco is chemically treated or processed to reduce the nicotine content. In still another aspect, the tobacco is produced from a genetically modified tobacco plant in which the tobacco plant has a genetic modification that causes an increased level of isomyosmine in the tobacco. In other aspects, the isomyosmine found in the tobacco is synthetic. In still other aspects, the tobacco has an isomyosmine content from about 0.001 mg/g to about 10 mg/g, from about 0.01 mg/g to about 10 mg/g, or from about 0.1 mg/g to about 100 mg/g. In still other aspects, the tobacco has a nicotine content of less than about 2 mg/g.

In accordance with another aspect, a tobacco plant is disclosed that produces tobacco with increased levels of isomyosmine due to a genetic modification in the tobacco plant. In accordance with another aspect, the tobacco plant also produces tobacco with reduced levels of nicotine. In other aspects, the reduced levels of nicotine in the tobacco produced by the tobacco plant are the result of a genetic modification that causes the plant to produce lower levels of nicotine in the tobacco. In yet other aspects, the tobacco is chemically treated or processed to reduce the levels of nicotine in the tobacco. In certain aspects, the genetically modified tobacco plant has a first genetic modification that induces the plant to produce tobacco with increased levels of isomyosmine, and a second genetic modification that inhibits the expression of the MTHFR gene. The inhibition of the expression of the MTHFR gene induces an increased expression of the CYP82E4 gene that results in an increased nicotine-to-nornicotine conversion rate. The increased nicotine-to-nornicotine conversion rate decreases the levels of nicotine in the tobacco.

### BRIEF DESCRIPTION OF THE DRAWINGS

A more complete understanding of the present invention and certain advantages thereof may be acquired by referring to the following detailed description in consideration with the accompanying drawings, in which:

FIG. 1 is a schematic representing the conversion of nicotine to nornicotine mediated by nicotine N-demethylase gene CYP82E4.

FIG. 2 is a schematic representing the molecular structures of several tobacco alkaloids.

### DETAILED DESCRIPTION

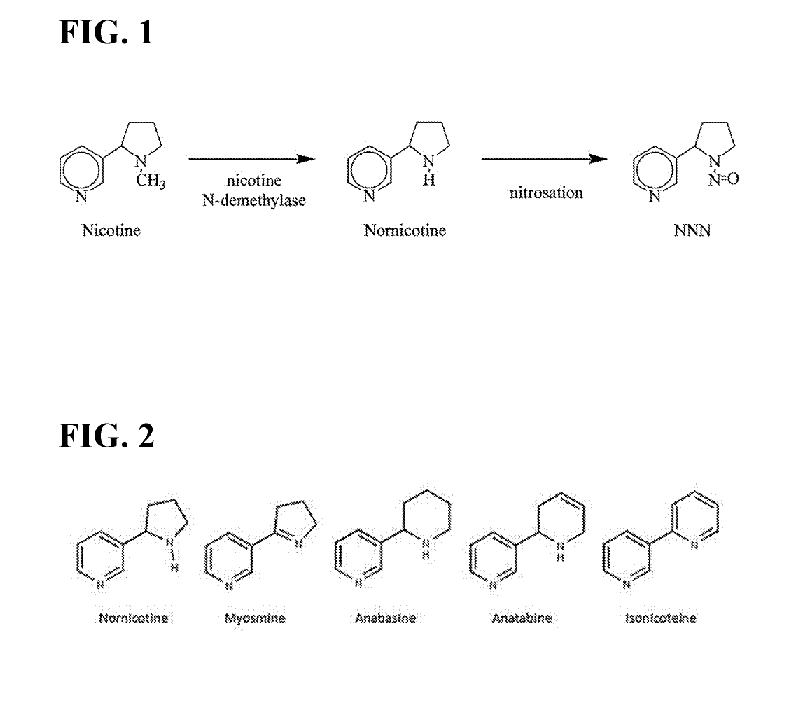

In tobacco plants, nicotine and other alkaloids are synthesized in the roots. The nicotine and other alkaloids are transported by the xylem to the leaf. Typically, 90% to 95% of the alkaloid content in commercial tobacco plants is nicotine, which is about 2% to 5% of dry leaf weight. Saitoh F, Nona M, Kawashima N (1985), The alkaloid contents of sixty Nicotiana species, Phytochemistry 24: 477-480. As depicted in FIG. 1, nicotine may undergo N-demethylation in the leaf to produce the alkaloid nornicotine. The biosynthesis of nornicotine from nicotine is largely mediated by the nicotine N-demethylase gene CYP82E4. Tobacco plants with a nicotine-to-nornicotine conversion rate (NCR) greater than about 3% have been defined as “convertors” and were previously held as commercially undesirable due to the reduced nicotine levels produced by the tobacco plant. Jack A, Fannin N, Bush L P (2007), Implications of reducing nornicotine accumulation in burley tobacco, Appendix A: the LC protocol. Rec Adv Tob Sci 33: 58-79.

According to another aspect, the tobacco disclosed herein has a nicotine content of, for example, at least, greater than, less than, equal to, or any number in between about 0.001 mg/g, 0.01 mg/g, 0.10 mg/g, 0.15 mg/g, 0.20 mg/g, 0.25 mg/g, 0.30 mg/g, 0.35 mg/g, 0.40 mg/g, 0.45 mg/g, 0.50 mg/g, 0.55 mg/g, 0.60 mg/g, 0.65 mg/g, 0.70 mg/g, 0.75 mg/g, 0.80 mg/g, 0.85 mg/g, 0.90 mg/g, 0.95 mg/g, 1.00 mg/g, 1.10 mg/g, 1.15 mg/g, 1.20 mg/g, 1.25 mg/g, 1.30 mg/g, 1.35 mg/g, 1.40 mg/g, 1.45 mg/g, 1.50 mg/g, 1.55 mg/g, 1.60 mg/g, 1.65 mg/g, 1.70 mg/g, 1.75 mg/g, 1.80 mg/g, 1.85 mg/g, 1.90 mg/g, 1.95 mg/g, 2.00 mg/g, 2.10 mg/g, 2.15 mg/g, 2.20 mg/g, 2.25 mg/g, 2.30 mg/g, 2.35 mg/g, 2.40 mg/g, 2.45 mg/g, 2.50 mg/g, 2.55 mg/g, 2.60 mg/g, 2.65 mg/g, 2.70 mg/g, 2.75 mg/g, 2.80 mg/g, 2.85 mg/g, 2.90 mg/g, 2.95 mg/g, 3.00 mg/g, 3.10 mg/g, 3.15 mg/g, 3.20 mg/g, 3.25 mg/g, 3.30 mg/g, 3.35 mg/g, 3.40 mg/g, 3.45 mg/g, 3.50 mg/g, 3.55 mg/g, 3.60 mg/g, 3.65 mg/g, 3.70 mg/g, 3.75 mg/g, 3.80 mg/g, 3.85 mg/g, 3.90 mg/g, 3.95 mg/g, 4.00 mg/g, 4.10 mg/g, 4.15 mg/g, 4.20 mg/g, 4.25 mg/g, 4.30 mg/g, 4.35 mg/g, 4.40 mg/g, 4.45 mg/g, 4.40 mg/g, 4.45 mg/g, 4.50 mg/g, 4.55 mg/g, 4.60 mg/g, 4.65 mg/g, 4.70 mg/g, 4.75 mg/g, 4.80 mg/g, 4.85 mg/g, 4.90 mg/g, 4.95 mg/g, 5.00 mg/g, 5.50 mg/g, 5.70 mg/g, 6.00 mg/g, 6.50 mg/g mg/g, 6.70 mg/g, 7.00 mg/g, 7.50 mg/g, 7.70 mg/g, 8.00 mg/g, 8.50 mg/g, 8.70 mg/g, 9.00 mg/g, 9.50 mg/g, 9.70 mg/g, 10.0 mg/g, 10.5 mg/g, 10.7 mg/g, and 11.0 mg/g, and at least, greater than, less than, equal to, or any number in between about 0.001 μg/g, 0.002 μg/g, 0.003 μg/g, 0.004 μg/g, 0.005 μg/g, 0.006 μg/g, 0.007 μg/g, 0.008 μg/g, 0.009 μg/g, 0.01 μg/g, 0.02 μg/g, 0.03 μg/g, 0.04 μg/g, 0.05 μg/g, 0.06 μg/g, 0.07 μg/g, 0.08 μg/g, 0.09 μg/g, 0.01 μg/g, 0.15 μg/g, 0.20 μg/g, 0.25 μg/g, 0.30 μg/g, 0.35 μg/g, 0.40 μg/g, 0.45 μg/g, 0.5 μg/g, 0.55 μg/g, 0.60 μg/g, 0.65 μg/g, 0.70 μg/g, 0.75 μg/g, 0.80 μg/g, 0.85 μg/g, 0.90 μg/g, 0.95 μg/g, 1.00 μg/g, 1.10 μg/g, 1.15 μg/g, 1.20 μg/g, 1.25 μg/g, 1.30 μg/g, 1.35 μg/g, 1.40 μg/g, 1.45 μg/g, 1.50 μg/g, 1.55 μg/g, 1.60 μg/g, 1.65 μg/g, 1.70 μg/g, 1.75 μg/g, 1.80 μg/g, 1.85 μg/g, 1.90 μg/g, 1.95 μg/g, 2.00 μg/g, 2.10 μg/g, 2.15 μg/g, 2.20 μg/g, 2.25 μg/g, 2.30 μg/g, 2.35 μg/g, 2.40 μg/g, 2.45 μg/g, and 2.50 μg/g.

Techniques for alteration of tobacco to reduce nicotine levels are known in the art and described, for example, in U.S. Patent Publication 2010/0206317, U.S. Pat. Nos. 3,901,248 and 6,907,887, the disclosures of which are hereby incorporated by reference in their entirety. Tobacco may be chemically treated to remove nicotine or a tobacco plant may be selectively bred to produce low alkaloid or low nicotine levels, such as tobacco plants having one or more mutations in genes encoding enzymes or proteins involved in nicotine biosynthesis. Other methods such as microbial enzymatic degradation, chemical extraction, or high pressure extraction may be employed to reduce nicotine levels in tobacco plants. More recently, techniques in genetic engineering and chemically induced gene suppression or expression have been used to reduce the nicotine levels in tobacco plants. Any one or more of these techniques can be used to create a tobacco or tobacco product used with the disclosures herein.

According to another aspect, a tobacco plant may be genetically modified to express, repress, alter, or mutate a gene or genes involved in nicotine and/or other alkaloid biosynthesis. According to yet another aspect, a genetically modified tobacco plant disclosed herein may produce low levels of nicotine and elevated levels of isomyosmine due to a modified gene or genes encoding an enzyme(s) or protein(s) involved in nicotine and/or isomyosmine biosynthesis.

A genetic engineering technique to decrease the nicotine content in a tobacco plant involves manipulating the methylenetetrahydrofolate reductase (MTHFR) gene in a tobacco plant. See Chiu-Yueh Hung, et al. (2013), Alteration of the Alkaloid Profile in Genetically Modified Tobacco Reveals a Role of Methylenetetrahydrofolate Reductase in Nicotine N-Demethylation, Plant Physiology February 2013, 161 (2) 1049-1060; DOI: 10.1104/pp. 112.209247. It is known that an altered MTHFR gene expression negatively regulates the expression of the nicotine N-demethylase gene CYP82E4. A genetically modified tobacco plant that suppresses the MTHFR gene induces the expression of the nicotine N-demethylase gene CYP82E4. Thus, a transgenic plan that suppresses the MTHFR gene favors nicotine N-demethylation in tobacco leaves and results in lower levels of nicotine production.

As shown in FIG. 2, a number of structurally related alkaloids are found in tobacco other than nicotine, including nicotine, nornicotine, myosmine, anabasine, anatabine, isonicoteine, and isomyosmine. Isomyosmine (3-(3,4-dihydro-2H-pyrrol-2-yl)-pyridine), shown below, is a nicotine related alkaloid present in solanecea plants containing nicotine.

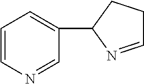 [SMILES: C1=NC(c2cccnc2)CC1]

In the examples in which isomyosmine is added to tobacco material, isomyosmine may be prepared synthetically using known techniques or obtained from chemical suppliers. As an alternative to synthetic preparation, isomyosmine may be obtained by extraction from tobacco or other materials in which it occurs naturally. For example, tobacco material may extracted with a solvent, such as water, ethanol, steam, and/or carbon dioxide. The resulting solution contains the soluble components of the tobacco, including alkaloids such as nicotine, isomyosmine, and myosmine. Isomyosmine may be purified using known techniques such as liquid chromatography. The purified isomyosmine may then be added to tobacco compositions or products.

According to another aspect, the tobacco disclosed herein has an isomyosmine content of, for example, at least, greater than, less than, equal to, or any number in between about 0.001 mg/g, 0.01 mg/g, 0.10 mg/g, 0.15 mg/g, 0.20 mg/g, 0.25 mg/g, 0.30 mg/g, 0.35 mg/g, 0.40 mg/g, 0.45 mg/g, 0.50 mg/g, 0.55 mg/g, 0.60 mg/g, 0.65 mg/g, 0.70 mg/g, 0.75 mg/g, 0.80 mg/g, 0.85 mg/g, 0.90 mg/g, 0.95 mg/g, 1.00 mg/g, 1.10 mg/g, 1.15 mg/g, 1.20 mg/g, 1.25 mg/g, 1.30 mg/g, 1.35 mg/g, 1.40 mg/g, 1.45 mg/g, 1.50 mg/g, 1.55 mg/g, 1.60 mg/g, 1.65 mg/g, 1.70 mg/g, 1.75 mg/g, 1.80 mg/g, 1.85 mg/g, 1.90 mg/g, 1.95 mg/g, 2.00 mg/g, 2.10 mg/g, 2.15 mg/g, 2.20 mg/g, 2.25 mg/g, 2.30 mg/g, 2.35 mg/g, 2.40 mg/g, 2.45 mg/g, 2.50 mg/g, 2.55 mg/g, 2.60 mg/g, 2.65 mg/g, 2.70 mg/g, 2.75 mg/g, 2.80 mg/g, 2.85 mg/g, 2.90 mg/g, 2.95 mg/g, 3.00 mg/g, 3.10 mg/g, 3.15 mg/g, 3.20 mg/g, 3.25 mg/g, 3.30 mg/g, 3.35 mg/g, 3.40 mg/g, 3.45 mg/g, 3.50 mg/g, 3.55 mg/g, 3.60 mg/g, 3.65 mg/g, 3.70 mg/g, 3.75 mg/g, 3.80 mg/g, 3.85 mg/g, 3.90 mg/g, 3.95 mg/g, 4.00 mg/g, 4.10 mg/g, 4.15 mg/g, 4.20 mg/g, 4.25 mg/g, 4.30 mg/g, 4.35 mg/g, 4.40 mg/g, 4.45 mg/g, 4.40 mg/g, 4.45 mg/g, 4.50 mg/g, 4.55 mg/g, 4.60 mg/g, 4.65 mg/g, 4.70 mg/g, 4.75 mg/g, 4.80 mg/g, 4.85 mg/g, 4.90 mg/g, 4.95 mg/g, 5.00 mg/g, 5.50 mg/g, 5.70 mg/g, 6.00 mg/g, 6.50 mg/g mg/g, 6.70 mg/g, 7.00 mg/g, 7.50 mg/g, 7.70 mg/g, 8.00 mg/g, 8.50 mg/g, 8.70 mg/g, 9.00 mg/g, 9.50 mg/g, 9.70 mg/g, 10.0 mg/g, 10.5 mg/g, 10.7 mg/g, and 11.0 mg/g.

The tobacco plant disclosed herein includes any plant of the genus Nicotiana of the nightshade family (Solanaceae). Another alternative to naturally produced tobacco related products, and/or tobacco plants with synthetic or naturally produced isomyosmine content is to engineer a transgenic solanecea plant that produces tobacco with elevated or increased levels of isomyosmine. It is known that tobacco plant metabolisms are controlled by genetic as well as environmental factors. A solanecea plant may be genetically modifying to increase the levels of isomyosmine in the tobacco leaves used in tobacco compositions. A solanecea plant may also be genetically modifying to decrease the levels of nicotine in the tobacco leaves used in tobacco compositions. A transgenic solanecea plant can be engineered to produce low levels of nicotine and/or elevated levels of isomyosmine due to a modified gene or genes encoding an enzyme(s) or protein(s) involved in nicotine and/or isomyosmine biosynthesis. According to another aspect, a solanecea plant may be genetically modified to express, repress, alter, or mutate a gene or genes involved in isomyosmine biosynthesis.

As used herein, the terms “protein” and “polypeptide” and “peptide” are used interchangeably herein to designate a series of amino acid residues, connected to each other by peptide bonds between the alpha-amino and carboxy groups of adjacent residues. The terms “protein,” “peptide,” and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “peptide” are used interchangeably herein when referring to a gene product and fragments thereof. Thus, exemplary polypeptides, peptides, or proteins include gene products, naturally occurring proteins, homologs, orthologs, paralogs, fragments and other equivalents, variants, fragments, and analogs of the foregoing.

According to one aspect, genes encoding a polypeptide related to isomyosmine biosynthesis may be utilized to overexpress or inhibit the expression of the polypeptide in a tobacco plant in which the polypeptide may normally be found. In other aspects, the gene may be used to design a polynucleotide that inhibits or induces the expression of the gene, and the polynucleotide may be introduced into a cell of the tobacco plant.

An “amino acid sequence” may be determined directly for a protein or peptide, or inferred from the corresponding nucleic acid sequence. A “nucleic acid” or “nucleic acid sequence” may be any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA. “Heterologous” as applied to nucleic acids is of different origin than that of the natural tobacco plant cell.

A “vector” refers to a piece of DNA, either single or double stranded. The vector can be for example, of plasmid or viral origin, which typically encodes a selectable or screenable marker or transgenes. The vector is used to transport the foreign or heterologous DNA into a suitable host cell. Once in the host cell, the vector can replicate independently of or coincidental with the host chromosomal DNA. Alternatively, the vector can target insertion of the foreign or heterologous DNA into a host chromosome.

The term “transgenic” refers to tobacco plant cells that have been “transformed.” “Transformed” describes the introduction of DNA into the tobacco plant cell. In most cases the DNA is introduced into the tobacco plant cell in the form of a vector containing the DNA segment. A transformed tobacco plant may be identified by selectable marker and report genes in accordance with methods known in the art. “Expressed” describes a protein that is produced in a plant cell when its DNA is transcribed to mRNA that is translated to the protein. “Inhibition” describes a measurable decrease in the cellular level of mRNA transcribed from the gene (i.e., coding polynucleotide), and/or in the cellular level of a peptide, polypeptide, or protein product of the coding polynucleotide. “Overexpression” describes a greater expression level of a gene in a tobacco plant or tobacco plant cell compared to the expression in a wild-type tobacco plant or cell. “Suppressed refers to decreased expression or activity of a protein.

The term “% sequence identity” describes the extent to which the sequences of DNA or protein segments are invariant throughout a window of alignment of sequences such as nucleotide sequences or amino acid sequences and is determined by comparing two optimally aligned sequences over a comparison window. An identity fraction for a sequence aligned with a reference sequence is the number of identical components which are shared by the sequences, divided by the length of the alignment not including gaps introduced by the alignment algorithm. “% identity” is the identity fraction times 100. “Substantially identical” describes nucleotide sequences that are more than 85% identical to a reference sequence.

“Promoter” describes a regulatory DNA that initializes transcription. Using methods known to a person of ordinary skill in the art, recombinant DNA constructs are assembled and usually include a promoter operably linked to DNA.

Definitions of common terms in cell biology and molecular biology can be found in The Encyclopedia of Molecular Biology, published by Blackwell Science Ltd., (1994) (ISBN 0-632-02182-9); Benjamin Lewin, (2009) Genes X, published by Jones & Bartlett Publishing, (ISBN-10: 0763766321); Kendrew et al. (eds.) (1995), Molecular Biology and Biotechnology: a Comprehensive Desk Reference, published by VCH Publishers, Inc., (ISBN 1-56081-569-8) and Current Protocols in Protein Sciences (2009), Wiley Intersciences, Coligan et al., eds.

Unless otherwise stated, the present disclosure is performed using standard procedures, as described, for example in Sambrook et al., (2001) Molecular Cloning: A Laboratory Manual (3 ed.), Cold Spring Harbor Laboratory Press, Cold Spring Harbor, N.Y., USA; Davis et al., (1995) Basic Methods in Molecular Biology, Elsevier Science Publishing, Inc., New York, USA; or Methods in Enzymology: Guide to Molecular Cloning Techniques Vol. 152, S. L. Berger and A. R. Kimmel Eds., Academic Press Inc., San Diego, USA (1987); and Current Protocols in Protein Science (CPPS) (John E. Coligan, et. al., ed., John Wiley and Sons, Inc.), which are all incorporated by reference herein in their entireties.

In some embodiments, tobacco plant genes encoding a polypeptide, related to the biosynthesis of isomyosmine, may be utilized to overexpress or inhibit the expression of the polypeptide in the tobacco plant in which the polypeptide is normally found. For example, the gene or a related vector may be introduced into a cell of the plant in a genetic locus where the gene is not normally found, or a native copy of the gene or related vector may be placed under regulatory control elements that lead to increased expression of the native gene or vector. In other examples, the gene may be used to design a polynucleotide that inhibits the expression of the gene, and the polynucleotide may be introduced into a cell of the tobacco plant. Accordingly, genetically modified tobacco plants may be engineered to increase or decrease the levels of certain alkaloids, such as isomyosmine, by overexpressing or inhibiting one or several key genes related to alkaloid biosynthesis levels.

In one example, a tobacco may be made with increased levels of isomyosmine by exposing a tobacco plant cell to a vector or exogenous DNA construct that includes a promoter that is operable in the tobacco plant cell and a DNA sequence capable of encoding an enzyme(s) or protein(s) critical to the isomyosmine alkaloid biosynthesis pathway inducing increased levels of isomyosmine production and biosynthesis. Accordingly, the tobacco plant cell is transformed by the DNA construct or vector. The transformed cells are selected and the resulting transgenic tobacco plant is regenerated using conventional techniques.

The expression of isolated nucleic acids encoding a protein involved in the isomyosmine biosynthesis pathway can be achieved by operably linking the DNA or cDNA to a promoter, and then incorporated into an expression vector. Vectors can be suitable for replication and integration in either prokaryotes or eukaryotes. Typical expression vectors contain transcription and translation terminators, initiation sequences, and promoters that regulate the expression of the DNA encoding a protein involved in the isomyosmine biosynthesis pathway. The vector is then introduced into the appropriate host cell.

In accordance with one aspect of the current disclosure, a genetically modified tobacco plant includes a tobacco plant transformation vector comprising a nucleic acid encoding a polypeptide having at least 80%, 85%, 88%, 90%, 92%, 95%, 98%, 99%, or 100% sequence identity to a tobacco plant gene related to enzymes, proteins, or other components critical to the isomyosmine alkaloid biosynthesis pathway, or directly related to the biosynthesis and/or the metabolic pathway of isomyosmine. In accordance with another aspect, the nucleic acid is operably linked to a promoter. In accordance with another aspect, a nucleic acid construct for genetically modified tobacco plants is provided. The nucleic acid construct comprises a polynucleotide sequence encoding a polypeptide having at least 80% sequence identity to a tobacco plant gene related to the isomyosmine alkaloid biosynthesis pathway, and one or more control sequences for driving expression of the polynucleotide sequence in the genetically modified tobacco plant.

Unless otherwise stated, “isomyosmine,” as used herein, refers to isomyosmine that has been prepared synthetically, isomyosmine that has been produced from natural materials in which it occurs, or isomyosmine that has been produced from a genetically modified tobacco plant. The amount of isomyosmine in a solid tobacco product composition usually ranges from about 0.001 mg/g to about 10 mg/g, from about 0.01 mg/g to about 10 mg/g, or from about 0.1 mg/g to about 100 mg/g. Desirable isomyosmine content in a tobacco product that is a solvent ranges from about 0.001 to about 10 mg/ml, often from about 0.01 to about 5 mg/ml, from about 0.05 to about 4 mg/ml, from about 0.1 to about 3 mg/ml, from about 0.2 to about 2.5 mg/ml, from about 0.3 to about 2 mg/ml, from about 0.2 to about 1.5 mg/ml, or from about 0.1 to about 1 mg/ml.

According to certain aspects, the transgenic tobacco plants, and related tobacco and tobacco products contain reduced nicotine levels that may reduce smoking behavior. Smoking behaviors may be reduced further and more effectively with the incorporation of increased isomyosmine levels as presently disclosed. Nicotine dependence may be reduced in current smokers and tobacco users, new users may be less likely to develop nicotine dependence and continue to smoke, and former smokers and tobacco users who lapse may be less likely to become regular users again. Unlike “light” cigarettes, very low nicotine content cigarettes contain substantially less nicotine in the tobacco. For example, the tobacco described in the present disclosure includes nicotine levels of less than 2 mg/g compared to 10-14 mg/g in a typical cigarette. Typical nicotine levels in other tobacco products, such as chewing tobacco or snuff, ranges from about 5-10 mg/g. Henningfield, J. E., Radzius, A., & Cone, E. J. (1995), Estimation of available nicotine content of six smokeless tobacco products. Tobacco Control, 4 (1), 57-61. E-cigarettes, assuming a series of 15 puffs is equivalent to smoking one cigarette, delivers about 0.025-0.77 mg nicotine, compared to one smoked tobacco cigarette of about 1.54-2.60 mg. Maciej L. Goniewicz, Ph.D., Tomasz Kuma, M. Pharm., Michal Gawron, M. Pharm., Jakub Knysak, M. Pharm., Leon Kosmider, M. Pharm.; Nicotine Levels in Electronic Cigarettes, Nicotine & Tobacco Research, Volume 15, Issue 1, 1 Jan. 2013, Pages 158-166, https://doi.org/10.1093/ntr/nts103.

In some aspects, tobacco product of the disclosure may include, but are not limited to, a cigarette, cigar, pipe tobacco, smokeless tobacco, chewing tobacco, capsule, tablet, or lozenge. According to other aspects, the tobacco products may be produced from the genetically modified tobacco plant of the present disclosure. According to other aspects, the modified tobacco of the present disclosure is suitable for conventional growing and harvesting techniques and the harvested tobacco leaves and stems are suitable for use in one or more of the tobacco products disclosed herein. In some aspects modified tobacco of the present disclosure can be processed and blended with conventional tobacco.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The following examples are set forth as being representative of the present disclosure. These examples are not to be construed as limiting the scope of the present disclosure as these and other equivalent embodiments will be apparent in view of the present disclosure, figures and accompanying claims.

What is claimed is:

1. A method of increasing a level of isomyosmine in a tobacco plant comprising
genetically modifying a tobacco plant with a genetic modification, wherein the genetic modification induces the tobacco plant to produce a tobacco with increased levels of isomyosmine in the tobacco; and
wherein the tobacco has decreased levels of nicotine.

2. The method of claim 1, wherein the genetically modified tobacco plant has a nicotine converter rate of about 3.0% or greater.

3. The method of claim 1, wherein the tobacco has a nicotine content of less than about 2 mg/g.

4. The method of claim 1, wherein the tobacco has an isomyosmine content of at least 0.1 mg/g.

5. The method of claim 1, wherein the tobacco has an isomyosmine content from about 0.001 mg/g to about 10 mg/g.

6. The method of claim 1, wherein the tobacco plant is a solanecea plant.

7. The method of claim 1, wherein the tobacco has decreased levels of nicotine due to a second genetic modification to the tobacco plant.

8. The method of claim 7, wherein the second genetic modification to the tobacco plant inhibits the expression of a methylenetetrahydrofolate reductase (MTHFR) gene.

9. The method of claim 8, wherein the inhibition of the expression of the MTHFR gene induces an increased expression of a nicotine N-demethylase (CYP82E4) gene.

10. The method of claim 9, wherein the increased expression of the CYP82E4 gene results in an increased nicotine-to-nornicotine conversion rate, and wherein the increased nicotine-to-nornicotine conversion rate decreases the levels of nicotine in the tobacco.

11. A tobacco comprising an increased level of isomyosmine, and a reduced level of nicotine.

12. A tobacco product comprising the tobacco of claim 11.

13. The tobacco product of claim 12, wherein the tobacco product is a cigarette, cigar, pipe tobacco, smokeless tobacco, chewing tobacco, capsule, tablet, or lozenge.

14. The tobacco of claim 11, wherein the tobacco is produced from a genetically modified plant, wherein the tobacco plant has a genetic modification causing a nicotine converter rate of about 3.0% or more.

15. The tobacco of claim 11, wherein the tobacco is processed, and wherein the processing reduces a nicotine content of the tobacco.

16. The tobacco of claim 11, wherein the tobacco is produced from a genetically modified tobacco plant, wherein the tobacco plant has a genetic modification causing an increased level of isomyosmine in the tobacco.

17. The tobacco of claim 11, wherein the isomyosmine is synthetic.

18. The tobacco of claim 11, wherein the tobacco contains an isomyosmine content of at least 0.1 mg/g.

19. The tobacco of claim 11, wherein the nicotine content is less than about 2 mg/g.

20. A genetically modified tobacco plant comprising
a genetic modification, wherein the genetic modification induces a tobacco plant to produce a tobacco with increased levels of isomyosmine; and
wherein the tobacco has decreased levels of nicotine.

21. The genetically modified tobacco plant of claim 20, wherein the tobacco plant has a second genetic modification, wherein the second genetic modification induces the tobacco plant to produce the tobacco with decreased levels of nicotine.

22. The genetically modified tobacco plant of claim 21, wherein the second genetic modification to the tobacco plant inhibits the expression of a MTHFR gene, wherein the inhibition of the expression of the MTHFR gene induces an increased expression of a CYP82E4 gene, wherein the increased expression of the CYP82E4 gene results in an increased nicotine-to-nornicotine conversion rate, and wherein the increased nicotine-to-nornicotine conversion rate decreases the levels of nicotine in the tobacco.

23. The genetically modified tobacco plant of claim 20, wherein the genetically modified tobacco plant has a nicotine converter rate of about 3.0% or greater.

24. The genetically modified tobacco plant of claim 20, wherein the tobacco has a nicotine content of less than about 2 mg/g.

25. The genetically modified tobacco plant of claim 20, wherein the tobacco has an isomyosmine content of at least 0.1 mg/g.
